(19) United States
(12) Patent Application Publication
(10) Pub. No.: US 2004/0028747 A1
(43) Pub. Date: Feb. 12, 2004

(54) Crystalline drug particles prepared using a controlled precipitation process

(76) Inventors: Christopher J. Tucker, Midland, MI (US); Sonke Svenson, Midland, MI (US); James E. Hitt, Midland, MI (US); Cathy A. Curtis, Midland, MI (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/213,956

(22) Filed: Aug. 6, 2002

(51) Int. Cl. A61K 51/00; A61K 9/14; A61K 9/16; A61K 9/50

(52) U.S. Cl. 424/497; 424/1.11

(57) ABSTRACT

Drug particles which are essentially crystalline and have a mean particle size below about 2 microns, when dispersed in water, are described. When added to an aqueous medium at 25-95% of the equilibrium solubility of the drug substance, the drug particles show complete dissolution, as characterized by a 95% reduction in turbidity, in less than 5 minutes. Using a controlled precipitation process to prepare such drug particles is also described. Such drug particles exhibit an enhanced dissolution rate and better stability as compared to particles prepared according to processes described in the prior art.

### FIELD OF THE INVENTION

The present invention relates to crystalline drug particles and in particular relates to crystalline drug particles prepared using a controlled precipitation process.

### DESCRIPTION OF PRIOR ART

High bioavailability and dissolution rates are desirable attributes of a pharmaceutical end product. Bioavailability is a term meaning the degree to which a pharmaceutical product, or drug, becomes available to the target tissue after being administered to the body. Poor bioavailability is a significant problem encountered in the development of pharmaceutical compositions, particularly those containing an active ingredient that is poorly soluble in water. Poorly water soluble drugs tend to be eliminated from the gastrointestinal tract before being absorbed into the circulation.

It is known that the rate of dissolution of a particulate drug can increase with increasing surface area, such as by decreasing particle size. Furthermore, crystalline drug particles are desirable because of the greater stability as opposed to amorphous particles. Efforts have been made to control the size and morphology of drug particles in pharmaceutical compositions. The most commonly employed techniques are precipitation and milling techniques.

U.S. Pat. No. 5,716,642 teaches the use of an acid-base precipitation method. However, the method described in the '642 patent results in a large concentration of salt which must be removed via dialysis in order to obtain relatively pure drug particles.

Examples of solvent precipitation methods are described in U.S. Pat. Nos. 4,826,689 and 6,221,398 B 1, in Hasegawa et al, “Supersaturation Mechanism of Drugs from Solid Dispersions with Enteric Coating Agents, Chem. Pharm. Bull. Vol. 36, No. 12, p. 4941(1988), and Frederic Ruch and Egon Matijevic, Preparation of Micrometer Size Budesonide Particles by Precipitation, Journal of Colloid and Interface Science, 229, 207-211 (2000). In the standard method described in these references, a supersaturated solution of the compound to be crystallized is contacted with an appropriate ‘anti-solvent’ in a stirred vessel. Within the stirred vessel, the anti-solvent initiates primary nucleation which leads to crystal formation. However, the crystals that are formed are relatively large, whereas the smaller particles described by these references are amorphous. For the relatively large crystalline particles, these methods almost always require a post-crystallization milling step in order to increase particle surface area and thereby improve their bioavailability. However, milling has drawbacks, including yield loss, noise and dust. Even wet milling techniques, as described in as described in U.S. Pat. No. 5,145,684, exhibit problems associated with contamination from the grinding media. Moreover, exposing a drug substance to excessive mechanical shear or exceedingly high temperatures can cause the drug to lose its activity. In addition, wet milling techniques always result in the presence of a fraction of larger particles, which affects the time for the particles to completely dissolve.

It would be an advantage in the art of preparation drug particles to provide particles which exhibit enhanced dissolution rates as compared with particles prepared according to methods described in the above prior art. It would also be an advantage if such particles were essentially crystalline in nature so as to minimize some of the problems associated with reduced stability of amorphous particles.

### SUMMARY OF THE INVENTION

In one aspect, the present invention is particles comprising a drug substance wherein the particles are essentially crystalline and have a mean particle size below about 2 microns when dispersed in water and wherein, when added to an aqueous medium at 25-95% of the equilibrium solubility of the drug substance, the drug particles show complete dissolution, as characterized by a 95% reduction in turbidity, in less than 5 minutes.

In a second aspect, the present invention is drug particles prepared according to a process comprising the steps of: (a) dissolving a drug substance in a solvent; and (b) adding the product of step (a) to water to form precipitated drug particles; wherein the drug particles are essentially crystalline and have a mean particle size below about 2 microns and wherein, when the drug particles are added to an aqueous medium at 25-95% of its equilibrium solubility, the drug particles show complete dissolution in less than 5 minutes.

The particles of the present invention exhibit a dissolution rate that is faster than particles prepared according to processes described in the prior art. The particles of the present invention are also essentially crystalline in nature, which results in a longer shelf life and better redispersibility as compared to particles that are amorphous in nature.

### BRIEF DESCRIPTION OF THE DRAWINGS

FIG. 1 is a picture showing the Scanning Electron Microscopy results for the particles prepared in Example 1.

FIG. 2 is a picture showing the Scanning Electron Microscopy results for the particles prepared in Example 4.

FIG. 3 is a graph depicting absorbance versus time for the particles of Examples 1 and 4.

FIG. 4 is a graph depicting percent material dissolved over time for the particles of Examples 1 and 4.

### DETAILED DESCRIPTION OF THE INVENTION

The particles of the present invention comprise a drug substance. In one embodiment, the drug substance is poorly soluble in water. Suitable drug substances can be selected from a variety of known classes of drugs including, for example, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiacinotropic agents, contrast media, corticosterioids, cough suppressants (expectorants and mucolytics), diagnostic agents, diagnostic imaging agents, diuretics, dopaminergics (antiparkinsonian agents), haemostatics, immuriological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasidilators and xanthines. Preferred drug substances include those intended for oral administration. A description of these classes of drugs and a listing of species within each class can be found in Martindale, The Extra Pharmacopoeia, Twenty-ninth Edition, The Pharmaceutical Press, London, 1989.

The drug particles of the present invention are essentially crystalline. As used herein the term “essentially crystalline” is defined to mean that the particles are at least 90% crystalline as measured using X-ray diffraction techniques.

The particles of the present invention are relatively small, especially after being dispersed in water. Preferably, the particles of the present invention have a mean particle size of less than about 2 microns when dispersed in water, more preferably less than about 1.5 microns, and even more preferably less than about 1.0 micron.

The particles of the present invention exhibit relatively fast dissolution rates. The preferred method for measuring dissolution rates for the particles of the present invention is a turbidity method. Turbidity gives a quantitative measurement of the change of intensity of light passing through a suspension of drug particles, caused by absorptive interactions resulting in energy transfer to the drug particles and by scattering from optical inhomogeneities in the drug particles. “Absorbance” is also a term that is used interchangeably with turbidity.

The turbidity method useful for determining the percent of dissolved material for the particles of the present invention comprises the following steps: determining the initial concentration of drug particles suspended in a liquid medium (i); determining the dynamic solid concentration (d) of drug particles in liquid medium; and calculating the percent dissolved material according to the formula: [(i−d)/i]×100. Turbidity measurements are used to determine (i) and (d).

Any liquid medium can be used, so long as the liquid medium is transparent in visible light and has a sufficiently different refractive index from the solid material such that it scatters light. The liquid medium should be chosen such that the equilibrium solubility of the drug particles in the liquid medium is between 5 and 500 mg/L. The term “equilibrium solubility” is defined herein to mean the maximum amount of drug particles that can be completely dissolved within 120 minutes in the liquid medium using this technique. To determine dissolution rates using turbidity measurements, one would need to develop a calibration curve showing turbidity versus a known concentration for the particular drug particles used. One would then measure the turbidity of the drug particles to be tested over time as the drug particles dissolve in the liquid medium, using commonly available light scattering equipment such as a calorimeter. One would then calculate i and d from the calibration curve based upon a measurement of turbidity. Such a method for measuring dissolution rates using turbidity is described in more detail in our copending U.S. patent application filed concurrently herewith.

When added to a liquid medium at a concentration that is from 25-95% of the equilibrium solubility, the particles of the present invention demonstrate complete dissolution in less than 5 minutes, as measured by the turbidity technique described above. The term equilibrium solubility is described above. More preferably, the drug particles can be added to the liquid medium at a concentration that is from 40-80% of their equilibrium solubility and still maintain complete dissolution in less than 5 minutes. As used herein, the term “complete dissolution” means that 95% of the particles are dissolved, as demonstrated by a 95% reduction in turbidity.

Optionally, one or more stabilizers are present in and on the surface of the particles of the present invention. Stabilizers can be used to inhibit substantial growth of the essentially crystalline particles, such that particles prepared in the presence of stabilizer are generally smaller than those prepared without a stabilizer.

The choice of stabilizer or stabilizers will depend upon the drug molecule. Generally, polymeric stabilizers are preferred. Examples of particle stabilizers include phospholipids, surfactants, polymeric surfactants, vesicles, polymers, including copolymers and homopolymers and biopolymers, and/or dispersion aids. Suitable surfactants include gelatin, casein, lecithin, (phospatides), gum acacia, cholesterol, tragacanth, stearic acid, benzalkonium chloride, calcium stearate, glyceryl monostearate, cetostearl alcohol, cetomacrogol 1000, polyoxyethylene castor oil derivatives, polyoxyethylene sorbitan fatty acid esters, e.g., the commercially available Tweens, polyethylene glycols, poly(ethylene oxide/propylene oxide) copolymers, e.g., the commercially available Poloxomers or Pluronics, polyoxyethylene stearates, colloidal silicon dioxide, phosphates, sodium dodecylsulfate, carboxymethlcellulose calcium, carboxymethylcellulose sodium, methylcellulose, hydroxyethylcellulose, hydroxypropylcellulose, hydroxypropylmethylcellulose, noncrystalline cellulose, magnesium aluminum silicate, triethanolamine, polyvinylalcohol, sodium lauryl sulfate, polyvinylpyrrolidone (PVP), poly(acrylic) acid, and other anionic, cationic, zwitterionc and nonionic surfactants. Other suitable stabilizers are described in detail in the Handbook of Pharmaceutical Excipients, published jointly by the American Pharmaceutical Association and The Pharmaceutical Society of Great Britain, the Pharmaceutical Press, 1986, which is incorporated by reference herein. Such stabilizers are commercially available and/or can be prepared by techniques known in the art.

Optionally, the particles of the present invention comprise one or more additional excipients which are added to the drug particles in order to enhance administration of the drug. Suitable additional excipients include polymers, absorption enhancers, solubility enhancing agents, dissolution rate enhancing agents, bioadhesive agents, and controlled release agents. More particularly, suitable excipients include cellulose ethers, acrylic acid polymers, and bile salts. Other suitable excipients are described in detail in the Handbook of Pharmaceutical Excipients, published jointly by the American Pharmaceutical Association and The Pharmaceutical Society of Great Britain, the Pharmaceutical Press, 1986, which is incorporated by reference herein. Such excipients are commercially available and/or can be prepared by techniques known in the art.

The particles of the present invention can be prepared using any method suitable for making small particles of poorly water soluble drug substances. In one aspect of the present invention, the particles are prepared by way of a controlled precipitation process. A “controlled precipitation process” is defined herein to mean a process comprising the following steps: (a) dissolving a drug substance in a solvent; and (b) adding the product of step (a) to water to form precipitated drug particles. In a preferred embodiment, an excipient, such as those described above, is present in the solvent, in the water or in both the solvent and the water.

The solvent into which the drug is dissolved in step (a) can be any organic solvent or water/organic solvent blend which dissolves the drug adequately. Generally, the higher the solubility of the drug in the solvent, the more efficient the process will be. The solvent should be miscible in water. Preferably, the selected solvent exhibits ideal mixing behavior with water so that the solution can be instantaneously distributed throughout the water when added to the water in step (b). Suitable organic solvents include but are not limited to methanol, ethanol, isopropanol, 1-butanol, t-butanol, trifluoroethanol, polyhydric alcohols such as propylene glycol, PEG 400, and 1,3-propanediol, amides such as n-methyl pyrrolidone, n,n-dimethylformamide, tetrahydrofuran, propionaldehyde, acetone, n-propylamine, isopropylamine, ethylene diamine, acetonitrile, methyl ethyl ketone, acetic acid, formic acid, dimethylsulfoxide, 1,3-dioxolane, hexafluoroisopropanol, and combinations thereof.

The concentration of drug dissolved in the solvent in step (a) is preferably as close as practical to the solubility limit of the solvent at room temperature. Such concentration will depend upon the selected drug and solvent but is typically in the range of from 0.1 to 20.0 weight percent.

In a preferred embodiment, the controlled precipitation process further comprises the step of mixing the product of step (b). Any external device which imparts intense mixing of the drug/solvent in the water can be used. “Intense mixing” is defined herein as meaning that a uniformly supersaturated mixture is formed prior to particle nucleation. The mixing should be sufficiently intense so as to result in nearly instantaneous dispersion of the drug/solvent solution across the water before new particle growth occurs. Such intense mixing results in supersaturation of the drug substance in the solvent and liquid mixture, causing drug particles to precipitate into small particles having a crystalline structure. Examples of devices which may be used to mix the product of step (b) include a stir bar, an agitator, a homogenizer and a colloid mill.

Optionally, the controlled precipitation process further comprises the step of recovering the precipitated drug particles. In one embodiment, recovering the drug particles comprises removing the solvent first and then subsequently removing the water. Alternatively, the solvent and water can be removed simultaneously from the particles. The choice will depend upon the concentration of solvent and the chosen method to remove the water. Removing the solvent can be performed using any desirable means including evaporation, dialysis and the like. Removing the water can be performed using any desirable means, including spray drying, spray freezing, gellation, (defined as gelling the particles with a polymer), lyophilization, or filtration. The method of solvent removal and water removal should be chosen such that when the resulting powder is dispersed in water, the particles are the same size and morphology as before the solvent and water are removed.

As the drug in organic solvent is added to the water in step (b), the temperature of the product of step(b) is optimally controlled at a reduced temperature. Preferably, the temperature is controlled at less than about 65° C., more preferably less than about 30° C., even more preferably less than about 23° C., and most preferably less than about 10° C. The lower limit of the temperature of the dispersion is the 0° C., the freezing point of water. Temperatures which are too high could lead to undesirable particle growth.

### EXAMPLES

The following materials were used in the following examples: “PVP” means 29,000 molecular weight polyvinylpyrrolidone. Examples 1 and 2: Particles of Danazol produced by way of a controlled precipitation process.

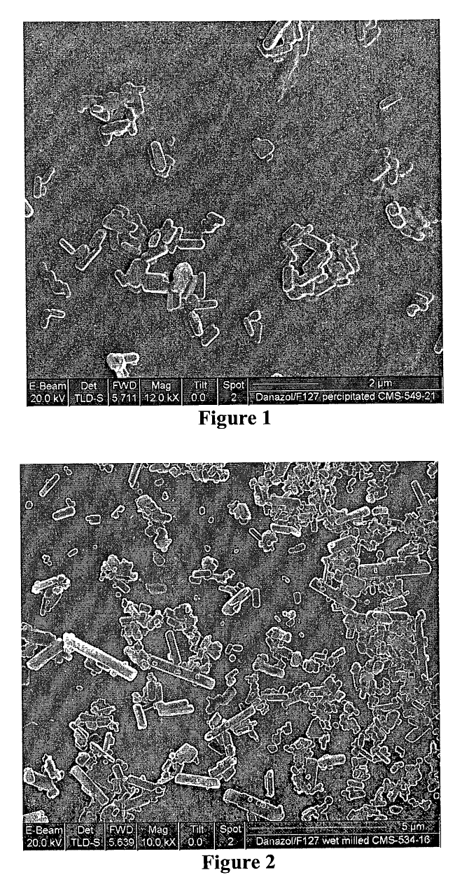

The following procedure is used to prepare particles by way of a controlled precipitation process. Table A lists the materials used and the results for Examples 1 and 2. 150 g of stabilizer in water (0.5 wt % for Example 1, 1.0 wt % for Example 2) was run through a recirculation loop and cooled to 4° C. To this aqueous solution was added 30 g of 0.5 wt % drug dissolved in methanol. This was repeated 18 times. The combined product was then twice run through a wiped film evaporator at 40° C. and a pressure of 26 mm Hg. The resulting slurry was then spray dried to a powder. The powder was then redispersed in water to 2% solids (followed by vortex agitation for ten seconds) and analyzed on a Coulter LS 230 particle size analyzer. X-ray diffraction patterns indicate that all samples are essentially crystalline. Scanning Electron micrographs (SEMs) confirm that the samples for Examples 1 and 2 are essentially crystalline and indicate that the particles have a narrow particle size distribution with no particles larger than 2 microns being observed. The SEM for Example 1 is shown in FIG. 1.

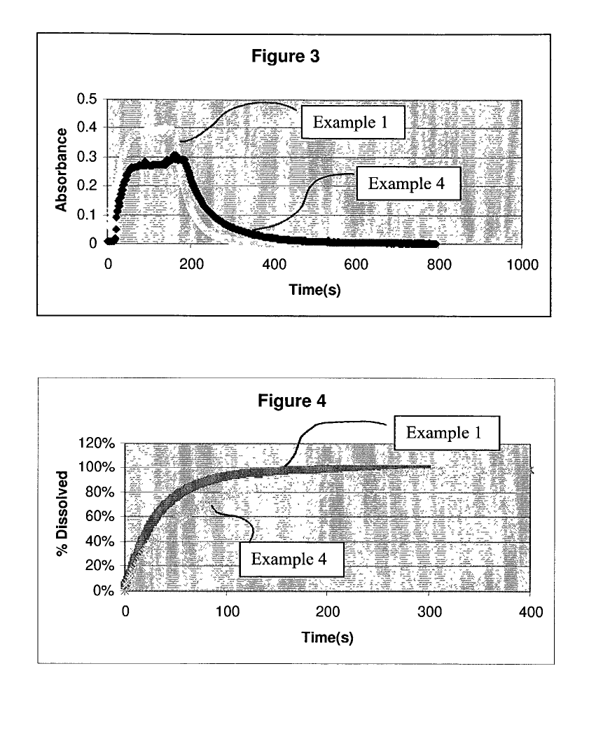

For each of Examples 1 and 2, to determine the time for complete dissolution, the following procedure utilizing turbidity measurements is followed. 5.1 mg of each sample was added to 150 ml of deionized water in a 200 ml plastic beaker equipped with a stir bar and a fiber optic turbidity probe (Brinkmann Colorimeter model PC-910 with a 450 nm light source filter and a 2 cm light path). The solubility of Danazol in water is approximately 1 mg per liter so one would expect approximately 0.22 mg of each sample to dissolve in 150 ml of water. After dispersing the sample for 150 seconds at a high stir rate the stir speed was turned down to 100 rpm and 2.25 g of 20% sodium dodecyl sulfate added. The addition of this amount of sodium dodecyl sulfate raises the equilibrium solubility of Danazol to approximately 45 mg/l. At this level the amount of each sample present correspond to 50.3% of the maximum amount of Danazol soluble in this media. Dissolution is then monitored by the loss in turbidity. FIG. 3 shows the results for Example 1 as compared to Example 4 described below.

Turbidity values can be transformed into percent dissolved by comparing the turbiditity at a given time to the average turbidity in the 10 seconds just prior to addition of the sodium dodecyl sulfate. If this is done and the time scales adjusted to zero seconds at the time of the sodium dodecyl sulfate addition one gets the dissolution curves shown in FIG. 4.

Results are indicated in Table A below.

### Example 3

### Particles of Naproxen Prepared Using a Controlled Precipitation Process

29.95 g of 6.69 wt % Naproxen in methanol was injected into 150.4 g of a 2 wt % solution of PVP in water at 2 degrees C. with vigorous stirring. The solvent was stripped from the resulting slurry and then freeze dried to yield a powder. Particle size and time to complete dissolution were measured as in Examples 1 and 2. Results are shown in Table A below.

TABLE A

| | | | | Time to complete |
|---|---|---|---|---|
| | | | Mean particle | dissolution |
| Ex. | Drug substance | Stabilizer | size (microns) | (seconds) |
| 1 | Danazol | Pluronic F127 | 0.54 | 111 |
| 2 | Danazol | Pluronic F127 | 0.29 | 78 |
| 3 | Naproxen | PVP | 0.24 | 31 |

### Examples 4 through 6

The following wet milling procedure was used to prepare the samples of Examples 4 through 6. Table B lists that materials used and the results. The stabilizer indicated in Table B was dissolved in water and placed in a wide mouth jar. To this was added the drug indicated in Table B below and a quantity of 1 mm ZrO milling beads, as indicated in Table B. The jar was then placed on a rotating ball mill and milled for the length of time indicated in Table B. The Jar was removed, the milling beads filtered off and the resulting slurry spray dried to a powder, which was then redispersed in water to 2% solids (followed by vortex agitation for ten seconds) and analyzed on a Coulter LS 230 particle size analyzer. The resulting particle sizes are shown in Table B. Scanning Electron Microscopy (SEM) results for Example 4 are shown in FIG. 2, which confirms that the particles are essentially crystalline but also shows that some particles larger than 2 microns are present.

The time to complete dissolution was measured as described above for Examples 1 through 3. Results are shown in Table B.

TABLE B

| | | | | | | | Mean | Time to |
|---|---|---|---|---|---|---|---|---|
| | | Stabilizer/ | | | Amount of | Milling | particle | complete |
| | | water | | Amount | milling | time | size | dissoln |
| Ex. | Stabilizer | (g/g) | Drug | drug (g) | beads (g) | (hrs) | (microns) | (sec) |
| 4 | Pluronic | 5.4/200 | Danazol | 10.8 | 1572 | 72 | 0.35 | 231 |
| | F127 | | | | | | | |
| 5 | Pluronic | 1.35/12 | Danazol | 1.35 | 100 | 39 | 0.35 | 128 |
| | F127 | | | | | | | |
| 6 | PVP | 1.35/12 | Naproxen | 1.35 | 100 | 48 | 0.26 | 67 |

FIG. 3 is a graph comparing absorbance over time as the drug particles of Examples 1 and 4 are dissolving. FIG. 4 shows the corresponding percent dissolved material for Examples 1 and 4 over time.

What is claimed is:

1. Particles comprising a drug substance wherein the particles are essentially crystalline and have a mean particle size below about 2 microns when dispersed in water and wherein, when added to an aqueous medium at 25-95% of the equilibrium solubility of the drug substance, the drug particles show complete dissolution in less than 5 minutes.

2. Particles according to claim 1 wherein the aqueous medium is chosen such that the equilibrium solubility of the drug substance is from 5 to 500 mg/L.

3. Particles according to claim 1 wherein complete dissolution is characterized by a 95% reduction in turbidity.

4. Particles according to claim 1 wherein the particles further comprise a stabilizer.

5. Particles according to claim 4 wherein the stabilizer is a phospholipid, surfactant, polymeric surfactant, vesicle, polymer, copolymer, homopolymer, biopolymer, dispersion aid or a combination thereof.

6. Particles according to claim 1 wherein the particles further comprise an excipient.

7. Particles according to claim 6 wherein the excipient is a polymer, absorption enhancer, solubility enhancing agent, dissolution rate enhancing agent, bioadhesive agent, controlled release agent, or a combination thereof.

8. Particles according to claim 1 wherein the drug substance is poorly water soluble.

9. Particles according to claim 8 wherein the drug substance is selected from the group consisting of analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiacinotropic agents, contrast media, corticosterioids, cough suppressants (expectorants and mucolytics), diagnostic agents, diagnostic imaging agents, diuretics, dopaminergics (antiparkinsonian agents), haemostatics, immuriological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasidilators, xanthines, and combinations thereof.

10. The particles according to claim 9 wherein the drug substance is intended for oral administration.

11. Drug particles prepared according to a process comprising the steps of:
(a) dissolving a drug substance in a solvent; and
(b) adding the product of step (a) to water to form precipitated drug particles;
wherein the drug particles are essentially crystalline and have a mean particle size below about 2 microns when dispersed in water and wherein, when the drug particles are added to an aqueous medium at 25-95% of its equilibrium solubility, the drug particles show complete dissolution in less than 5 minutes.

12. Drug particles according to claim 11, wherein the aqueous medium is chosen such that the equilibrium solubility of the drug substance is from 5 to 500 mg/L.

13. Drug particles according to claim 11, wherein the complete dissolution is characterized by a 95% reduction in turbidity.

14. Drug particles according to claim 11, wherein one or more stabilizers are present in the solvent, in the water, or in both the solvent and the water.

15. Drug particles according to claim 14, wherein the stabilizer is a phospholipid, surfactant, polymeric surfactant, vesicle, polymer, copolymer, homopolymer, biopolymer, dispersion aid or a combination thereof.

16. Drug particles according to claim 11, wherein one or more excipients are present in the solvent, in the water, or in both the solvent and the water.

17. Drug particles according to claim 16, wherein the excipient is a polymer, absorption enhancer, solubility enhancing agent, dissolution rate enhancing agent, bioadhesive agent, controlled release agent, or a combination thereof.

18. Drug particles according to claim 11, wherein the drug substance is poorly water soluble.

19. Drug particles according to claim 18, wherein the drug substance is selected from the group consisting of analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiacinotropic agents, contrast media, corticosterioids, cough suppressants (expectorants and mucolytics), diagnostic agents, diagnostic imaging agents, diuretics, dopaminergics (antiparkinsonian agents), haemostatics, immuriological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasidilators, xanthines, and combinations thereof.

20. Drug particles according to claim 11, wherein the process further comprises the step of mixing the product of step (b).

21. Drug particles according to claim 11, wherein the process further comprises the step of drying the precipitated drug particles.

22. Drug particles according to claim 11, wherein step (b) is performed at less than about 65° C.
